(19) United States
(12) Patent Application Publication
(10) Pub. No.: US 2024/0398685 A1
(43) Pub. Date: Dec. 5, 2024

(54) A TOPICAL COMPOSITION FOR PROVIDING ENERGIZING GLOW ON SKIN

(71) Applicant: Conopco, Inc., d/b/a UNILEVER, Englewood Cliffs, NJ (US)

(72) Inventors: Anita DAMODARAN, Bangalore (IN); Aditi Jayavant KULKARNI, Munbai (IN); Satish Kumar VENKATESH, Karnataka (IN); Mruthyunjaya Swamy MATHAPATHI, Bangalore (IN); Ankita Rutu PAWAR, Mumbai (IN); Tanya Rachael THOMAS, Bangalore (IN)

(73) Assignee: Conopco, Inc., d/b/a UNILEVER, Englewood Cliffs, NJ (US)

(21) Appl. No.: 18/696,127

(22) PCT Filed: Sep. 28, 2022
(86) PCT No.: PCT/EP2022/076892

§ 371 Date: Mar. 27, 2024

(30) Foreign Application Priority Data
Oct. 7, 2021 (EP) 21201525.9

(51) Int. Cl. A61K 8/67; A61K 8/96; A61Q 19/00

(52) U.S. Cl. CPC A61K 8/676; A61K 8/965; A61Q 19/00

(57) ABSTRACT

The present invention relates to a topical composition that provides energizing glow to skin. It more particularly relates to a personal care composition for topical application that delivers a fresh, young and lively look to skin. This is achieved through a combination of Olivine extract and a vitamin C compound.

### FIELD OF THE INVENTION

The present invention relates to a topical composition that provides energizing glow to skin. It more particularly relates to a personal care composition for topical application that delivers a fresh, young and lively look to skin

### BACKGROUND OF THE INVENTION

People in general like to look good and thereby feel good about themselves. In addition to eating good food and living a healthy lifestyle that includes breathing clean air and having balanced amount of exercise, they also rely on cosmetic products to keep their external appearance as close to their perception of what good looks constitute. Skin is the largest organ in the human body and the one that is most visible to the external world. In skin, most consumers want to have an even skin tone that is free of blotches, hyperpigmentation, wrinkles and visible signs of acne. Very many cosmetic products are available to deliver one or more of the above benefits. Many of the above problems are exacerbated by natural aging and internal and external stresses like light, excessive sunshine, pollution, hormonal changes and mental stress. The present inventors have worked on providing solutions to some of the above problems and developed technologies that deliver even skin tone, skin brightness and products that mask visible skins of aging like wrinkles.

Energizing brightness is another one of the benefits that some products in the market have been providing. Energizing brightness delivers a fresh and young look to skin, makes the skin lively, recharges the skin and helps protect against external and internal stresses. There are actives available in the market that are claimed to deliver this benefit e.g. Oli′Vine™ ST a product sold by Gattefosse. It has the INCI name Water (and) Olivine Extract. It is claimed that this active when applied to skin boosts cell metabolism and reduces the effects of stress. It is marketed as an energizing mineral complex which has magnesium as an active ingredient which helps tired and stressed skin to regain vitality and restore its energetic balance. They have shown that use of this active increases ATP (adenosine triphosphate) levels and reduces intracellular oxidation which are the key processes that deliver the claimed benefits to the skin.

Olivine has been used in cosmetic products in the past. US20200376024 (Teikoku Co. Ltd) discloses an external composition for skin including a rhodochrosite extract extracted from rhodochrosite with water, a hematite extract extracted from hematite with water, a smithsonite extract extracted from smithsonite with water, and an olivine extract. This has been claimed to deliver enhanced body balance even for ordinary people who do not aggressively exercise on a routine basis other than athletes.

CN104784071 (2015 by Guangdong Lifeng Cosmetics Making Co. Ltd) discloses a skin comprehensive anti-aging water condensation and a preparation method thereof. The water condensation is prepared from a hydroxyethyl acrylate/sodium acryloyldimethyl taurate copolymer, hydrogenated lecithin, xanthan gum, jellyfish collagen, jojoba oil, vitamin E, a hematite extraction liquid, an olivine extraction liquid, a rhodochrosite extraction liquid, a malachite extraction liquid, a smithsonite extraction liquid, phenoxyethanol, ethylhexylglycerin, algisium C and water. The anti-aging water condensation has the main effective components being the liquid ore extracts rich in various trace elements. The anti-aging water condensation can prolong the life of cells and delay skin aging, and is a comprehensive nursing type product which can comprehensively deal with the skin aging problem.

Thus, compositions comprising Olivine extract along with various other ingredients are disclosed that claim to deliver the inherent benefits of olivine. The present inventors too wished to deliver such a benefit using minimal amount of such actives which are generally expensive and therefore would add cost to the consumer. When Olivine extract was formulated by the present inventors in a simple aqueous composition that was made using water and certain thickening polymers, they found that when higher amount of Olivine extract was included in the composition, the desired energizing glow was delivered to the skin (as evident in increased ATP levels in HDFa cells), however the composition developed an undesirable green colour over time. When the concentration of Olivine was decreased the colour problem reduced but the desired benefit was not obtained. Thus, the present inventors were left with a problem as to how to deliver the desired energizing glow to skin through a composition which does not undesirably colour.

After using several actives suggested in the literature, they found that none of them could meet all of the required criteria set by the inventors like low cost, sufficient colour stability, formulation stability, and action standards on the required level of energizing brightness. They then found to their surprise that a relatively well known and easily available material which is not known for this benefit viz. vitamin C (which is available in the form of sodium ascorbyl phosphate) interacted synergistically with Olivine extract (at low concentrations) to deliver the contrasting requirements of enhanced ATP levels and low colour to the formulation. They also found that Vitamin C also boosts ATP levels which is a finding, to the knowledge of the present inventors, not known heretofore.

It is thus an object of the present invention to deliver energizing glow to skin from a stable topical composition.

It is another object of the present invention to deliver above benefit at low cost.

### SUMMARY OF THE INVENTION

The first aspect of the present invention relates to a topical composition for providing energizing glow to skin comprising olivine extract and vitamin C or its derivative; and a cosmetically acceptable vehicle selected from (i) water thickened with a polymer or (ii) an emulsion.

Another aspect of the present invention relates to a method of providing energizing glow to skin comprising the step of applying the composition of the invention on to skin.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The topical composition of the invention is meant to be used for personal care or for cosmetic use and could also be referred to as a personal care composition or a cosmetic composition. By a “personal care composition” as used herein, is meant to include a composition for topical application i.e external surfaces of the skin and/or hair of humans. Such a composition may be classified as leave-on or rinse off, and includes any product applied to a human body for improving appearance, cleansing, odour control or general aesthetics. The composition is preferably of the leave-on type. The composition of the present invention can be in the form of a liquid, lotion, cream, foam, stick, serum, essence or gel. Non-limiting examples of such compositions include leave-on gels, lotions, serum, essence or creams, preferably it is in the serum or essence form.

Serums are normally considered to be high concentration/potent leave-on products with actives/skin beneficent ingredients. Essence on the other hand is a water-based composition containing active ingredients to hydrate and protect the skin. Both the formats are meant to be used for easy absorption and deep penetration of actives into skin layers.

“Skin” as used herein is meant to include skin on the face and body (e.g. neck, chest, back, arms, underarms, hands, legs and scalp) and especially to the exposed parts thereof.

The composition of the invention comprises olivine extract which has the INCI name Water (and) Olivine Extract. This is available as Oli′Vine™ ST from Gattefosse, France. It is extracted from stones and so is of mineral origin. It is usually found in South Africa and the USA. The mineral is also known as peridot or chrysolite. In lithography, it is known as an energizing stone for stabilizing emotions. It contains magnesium and iron silicates. The extract is generally prepared using a unique extraction process which involves steps like grinding the stone to a powder, extraction with a specific chemicals, the extract thereafter stabilized to make the trace elements (in this case predominantly magnesium) more bioavailable for cosmetic application. The process then comprises the steps of filtration and sterilization to prepare a liquid stone extract.

The Olivine extract is claimed to boost cell metabolism, reduce effects of stress and acts on keratinocytes and fibroblasts. It is reported in the literature that stress causes metabolic imbalance which affects the vitality of the cells. Stress also causes reduced cell respiration which leads to reduced ATP production in mitochondria. Olivine extract has been found to reverse this where through oxygraphy test and measurement of ATP level on human keratinocytes and fibroblasts, it is found to boost cellular respiration and cell energy level. Other tests on measurement of mitochondrial membrane potential with stress inducers like FCCP, indicate that Olivine extract regulates the cell adaptive answer which is a sign of reduced stress. It is also reported to modulate the production of ROS and therefore limit the aging process in case of mitochondrial stress which is determined through cellular oxidation as measured using fluorescence intensity and senescence, and evaluated through counting of β-galactosidase positive cells.

Olivine extract is preferably included in 0.01 to 3%, more preferably 0.1 to 2% by weight of the composition.

The composition comprises vitamin C or a derivative thereof. Vitamin C (ascorbic acid, ascorbate) is a simple low-molecular-weight carbohydrate that is essential for the body as a water-soluble vitamin. As an antioxidant, vitamin C has both oxidized and reduced forms: L-dehydroascorbic and L-ascorbic acid.

Vitamin C is involved in the formation of the skin barrier and collagen in the dermis and plays a physiological role in the skin against skin oxidation, in antiaging of wrinkles, and in cell signal pathways of cell growth and differentiation, which are related to the occurrence and development of various skin diseases.

Vitamin C or its derivatives which may be used in the present invention may be one or more of the following:

### Sodium Ascorbyl Phosphate:

Sodium ascorbyl phosphate is an active stable vitamin C derivative ingredient for the cosmetics industry. It liberates vitamin C in the skin and protects the cells of the skin, promotes collagen formation, controls the formation of senile keratosis, and lightens dark skin.

### 3-O-Ethyl Ascorbic Acid:

3-O-ethyl ascorbic acid promotes the production of collagen, which significantly improves the structure of the skin cells and the overall condition of skin.

### Ascorbyl Methylsilanol Pectinate:

It is a known antioxidant and is known to improve skin texture, as well as helping with reducing aging and dark spots.

### Magnesium Ascorbyl Phosphate (MAP):

Magnesium ascorbyl phosphate (MAP) has all the functions of vitamin C and is very stable. It has excellent anti-oxidation and protection benefits, and can effectively resist UV radiation and promotes collagen production. This product is recommended for skin whitening/lightening applications, as well as anti-aging and anti-wrinkle products.

### Ascorbic Acid:

Vitamin C (L-ascorbic acid), USP is an active form of vitamin C as it occurs naturally. It is a potent antioxidant (shown to be able to protect skin from oxidative damages). It can improve appearance of aged and fragile skin. Widely used as add-on ingredient in skin-lightening products to correct hyperpigmentation and age spots.

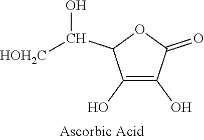 [SMILES: O=C1OC(C(O)CO)C(O)=C1O]

### Ascorbyl Glucoside:

Ascorbyl glucoside is a water-soluble Vitamin C derivative with superior stability. It resists degradation, and has the same lightening, sun protective and anti-aging properties as ascorbic acid.

### Tetrahexyldecyl Ascorbate:

Tetrahexyldecyl ascorbate is a very stable, oil-soluble Vitamin C ester but has no inherent capabilities as an antioxidant because all hydroxyl groups are esterified.

It has excellent skin penetration and as a result offers increased cell protection against UV-B radiation. It can improve appearance of aged and fragile skin. It is widely used as an add-on ingredient in skin-lightening products to correct hyperpigmentation and age spots.

### Aminopropyl Ascorbyl Phosphate:

It is a stabilized vitamin C derivative. It is ideal for whitening, anti-wrinkle, and anti-oxidation applications.

Vitamin C or its derivative is preferably included in 0.01 to 2%, more preferably 0.05 to 1.5% by weight of the composition.

The present inventors do not fully understand the reason behind the synergistic interaction between Olivine extract and Vitamin C or its derivative to deliver the benefits of the present invention. The extent of scientific understanding, by the present inventors, on these actives and their physiological activity is summarized below.

Olivine ST is a mineral extract rich in magnesium and many enzymatic activities are dependent on magnesium [Mg2+]. Magnesium plays an important role in maintaining energy supply in cells, and its dysregulation causes cellular malfunction and reduces mitochondrial ATP production.

Since, olivine extract is rich with magnesium, it stimulates the cell metabolic activity, acting both on cellular respiration and on the synthesis of ATP.

Mitochondria is known as a powerhouse of the cell as this produces bulk of the cellular ATP. It is known that many external factors like UV, pollution etc., induce reactive oxygen species (ROS) in the cell leading to oxidative stress. The excessive reactive oxygen species causes cellular damage and impacts on mitochondrial functions, thus reducing ATP synthesis. In addition, the reactive oxygen species like superoxide, singlet oxygen, peroxides are also produced during the process of cellular respiration and metabolism under normal condition which could also have undesirable consequences if not quenched.

Antioxidants play a very important role in reducing oxidative stress induced damages in the cell by quenching ROS. Vitamin C is one such antioxidant and has been demonstrated to have potent activity in scavenging ROS. We hypothesise that vitamin C by its antioxidant mechanism negates the effects of oxidative stress which in turn improves the ATP status of the cells.

The composition of the invention comprises a cosmetically acceptable vehicle selected from (i) water thickened with a polymer or (ii) an emulsion. The composition may be delivered in a lotion, cream, serum, essence or gel form, of which serum, essence or gel form is more preferred, the serum and essence form is most preferred. To enable this, the composition may comprises a cosmetically acceptable vehicle. The cosmetically acceptable vehicle is preferably chosen from water (which maybe thickened using polymers), or an emulsion which may be water-in-oil or an oil-in-water emulsion. Products in cream or lotion form are generally emulsions.

All of these product forms generally have a high content of water which may be in the range of 50 to 99%, preferably 70 to 95%, more preferably 85 to 95% by weight of the composition.

Products in the gel form contain predominantly water in the range of 65 to 90 wt %, in serum in 60 to 90 wt % and in essence in 85 to 95 wt % of the composition.

The most preferred polymer which acts as a thickener in water is an acrylate/alkyl acrylate cross polymer. These are sold under the brand names Pemulene TR-1, Pemulene TR-2, Ultrez 20, Ultrez 21. Other polymers which may be included are crosslinked acrylates (e.g. Carbopol 982), hydrophobically-modfied (e.g. carbopol 1382), carbomer and ammonium acryloyldimethyltaurate/beheneth-25 methacrylate crosspolymer, ammonium acryloyldimethyltaurate/VP copolymer, cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. The most preferred thicker is selected from one or more of acrylate/alkyl acrylate cross polymer, carbomer, ammonium acryloyldimethyltaurate/beheneth-25 methacrylate crosspolymer, ammonium acryloyldimethyltaurate/VP copolymer. Concentration of the thickener in the composition may range from 0.0001 to 5%, usually from 0.001 to 1%, optimally from 0.01 to 0.5% by weight.

The composition of the invention may include a humectant which is generally a polyhydric alcohol. Preferably the humectant is at least one of propylene glycol, dipropylene glycol, polypropylene glycol, polyethylene glycol, sorbitol, hydroxypropyl sorbitol, hexylene glycol, 1,3-butylene glycol, isoprene glycol, 1,3 propanediol, pentylene glycol, hexylene glycol, 1,2,6-hexanetriol, glycerol, ethoxylated glycerol, and propoxylated glycerol. Preferred humectants are selected from one or more of glycerine, 1,3-butylene glycol, propylene glycol, 1,3 propanediol, pentylene glycol, hexylene glycol, and sorbitol. Glycerol is the most preferred humectant. It is preferred that the amount of humectant is 1 to 10%, more preferably 2 to 8% by weight of the composition.

The composition preferably includes a skin brightening compound. Illustrative substances are placental extract, lactic acid, vitamin B3 compound preferably niacinamide, arbutin, kojic acid, ferulic acid, hydroquinone, resorcinol and derivatives including 4-substituted resorcinol (e.g. hexyl resorcinol or ethyl resorcinol), hydroxystearic acid (HSA) preferably 12-HSA, pyridoxine hydrochloride, alpha hydroxyacid (AHA), beta hydroxyacid (BHA), polyhydroxy acid (PHA) or natural skin brightening extracts like honey or lemon extracts and combinations thereof. More preferably, such skin brightening compound is selected from one or more of vitamin B3 compound, resorcinol like ethylresorcinol or hexylresorcinol, hydroxystearic acid, pyridoxine hydrochloride, AHA, BHA, PHA or natural skin brightening extracts like honey or lemon extracts. Most preferably the skin brightening compound is selected from one or more of niacinamide, 4-substituted resorcinol (e.g. 4-ethyl resorcinol or 4-hexyl resorcinol) and 12-HSA. Amounts of these skin brightening compounds may range from 0.01 to 10%, preferably from 0.1 to 2% by weight of the composition.

A preferred format for the solid form of the composition is a cream, further more preferably one which has a vanishing cream base. Vanishing cream base is one which comprises 3 to 25 wt % fatty acid. Optionally, the composition may comprise 0.1 to 10 wt % soap. When included, the fatty acid is preferably a C10 to C22 fatty acid, more preferably a C16 to C18 fatty acid. Most preferably the fatty acids are stearic acid or palmitic acid or a mixture thereof and the soap is preferably the potassium salt of the fatty acid mixture. The fatty acid is often hystric acid which is substantially (generally about 90 to 95%) a mixture of 45% stearic acid and 55% palmitic acid.

Preferably, the composition comprises emollients. Examples of emollients that may be used in the leave-on composition include stearyl alcohol, glyceryl monoricinoleate, mink oil, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the composition comprises solvents. Examples of solvents that may be used in the composition include ethyl alcohol, isopropanol, acetone, ethylene glycol ono ethyl ether, diethylene glycol mono butyl ether, diethylene glycol mono ethyl ether and mixtures thereof.

Preferably, the composition comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the composition comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C₆-C₁₂ alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred. When present in the composition, preservatives are added preferably in an amount 0.001 to 5 wt %, more preferably 0.01 to 3 wt % and most preferably 0.02 to 2 wt %, even most preferably 0.25 to 1.5%.

Preferably, the composition comprises a range of other optional ingredients that include antioxidants, binders, buffering agents, colorants, astringents, fragrance, opacifying agents, conditioners, exfoliating agents, pH adjusters, skin sensates, skin soothing agents, and skin healing agents.

The composition of the invention could also be incorporated in a format for cleansing topical surfaces of the human or animal body. Cleansing composition are of the so called wash-off or rinse-off format. By these formats, is meant that the composition is generally diluted with water and applied on the topical surface e.g. the skin or hair and then rinsed off with copious amounts of water after a few minutes. Cleansing composition could be in any format either in solid form or in liquid form. When in solid form it is preferably a bar which may contain soap, synthetic anionic surfactant or a combination of the two.

The soap for preparing the cleansing composition of the invention is preferably a C₈-C₂₄ soap, more preferably C₁₀-C₂₀ soap and most preferably C₁₂-C₁₈ soap. The cation of the soap can be alkali metal, alkaline earth metal or ammonium. Preferably, the cation of the soap is selected from sodium, potassium or ammonium. More preferably the cation of the soap is sodium or potassium. Fatty acids derived from other suitable oils/fats such as groundnut, soybean, tallow, palm, palm kernel, etc. may also be used in other desired proportions.

Synthetic anionic surfactants for use in wash-off compositions are preferably selected from alkyl ether sulphate, primary alkyl sulphate, secondary alkyl sulphonates, alkyl benzene sulphonates, or ethoxylated alkyl sulphates. The anionic surfactant other than soap which is preferred in the cleansing composition is an alkyl ether sulphate preferably those having between 1 and 3 ethylene oxide groups, either from natural or synthetic source and/or sulphonic acid. Especially preferred are sodium lauryl ether sulphates. Alkyl polyglucoside may also be present in the composition, preferably those having a carbon chain length between C₆ and C₁₆.

When present, the anionic surfactant is preferably present in an amount of 1 to 90%, preferably from 10 to 85%, more preferably 25 to 75% by weight of the cleansing composition. The cleansing composition is preferably in the form of a solid or semi solid form, most preferably in a solid form. Preferred solid compositions are in the shape of a soap bar.

Preferred solid cleansing compositions may include other known ingredients such as perfumes, pigments, preservatives, emollients, sunscreens, gelling agents and thickening agents. Water is a preferred carrier. When water is present, it is preferably present in at least 1%, more preferably at least 2%, further more preferably at least 5% by weight of the composition. When water is the carrier, a preferred cleansing composition comprises 10 to 50%, more preferably 12 to 40%, most preferably 12 to 22% by weight water.

The cleansing composition of the invention may also be delivered through a moisturizing bar or a moisturizing liquid composition. Moisturizing bar compositions comprising fatty acyl isethionates (e.g. cocyl isethionate) are especially preferred. Fatty acyl isethionates (e.g., cocoyl isethionates) surfactant “products” are defined as mixtures of anionic acyl isethionate surfactants and fatty acids/fatty acid soaps. They are highly desirable in personal care skin or hair cleansing products, particularly in personal care products, because they lather well, are mild to the skin and have good emollient properties. Typically, fatty acid isethionate surfactant products are produced by esterification of fatty acids or by reaction of fatty acid chloride having carbon chain length of C8 to C20 with isethionate. A typical surfactant product containing fatty acyl isethionate contains about 40 to 95 wt. % acid isethionate, and 5 to 50 wt. %, typically 10 to 40 wt. % free fatty acid, in addition to isethionate salts, typically at less than 5%, and trace (less than 2 wt. %) of other additives. Fatty acid soap may be included in the range of 5 to 15 wt %. Other surfactants like betaines may be included in 1 to 5 wt %. Water is generally included in 2 to 8 wt % of the composition.

Wash-off composition in the form of a liquid generally comprises surfactants at low concentration and are mild on skin. The surfactant is generally included in 4 to 18%, preferably 6 to 12% by weight of the liquid cleansing composition. Surfactants for inclusion in the liquid cleansing composition of the invention may preferably be of the anionic, non-ionic, cationic or amphoteric types. A useful surfactant for inclusion in the liquid cleansing composition of the invention is sodium lauryl ether sulphate (SLES). The SLES for use in the present invention generally preferably has 1 to 3 Ethoxylate (EO) groups. SLES is preferably included in 3 to 8% by weight of the composition. The other surfactant which may be included in the present invention is cocoamide monoethanol amine (CMEA). CMEA is preferably included in 1 to 3% by weight of the composition. One preferred aspect of the liquid cleansing composition of the invention relates to an aspect wherein the surfactant comprises a mixture of sodium lauryl ether sulphate (SLES) and coco amide monoethanol amine (CMEA). Another useful surfactant for inclusion in the liquid cleansing composition of the invention is an amphoteric surfactant preferably a betaine surfactant, more preferably an alkyl amidopropyl betaine surfactant for example cocamidopropyl betaine. In a preferred embodiment, the composition comprises from 0.1 to 5 wt. %, preferably from 0.5 to 4 wt. %, more preferably from 1 to 3 wt. % of a betaine surfactant.

Surfactants of the non-ionic class for inclusion in the liquid cleansing composition of the invention are preferably of the polyoxyethylene sorbitan alkyl esters class (sold as Tween surfactants)), fatty alcohol ethoxylates (sold as Brij surfactants), alkyl phenol ethoxylates (sold as Triton surfactants), fatty acid ethoxylates (sold as Myrj surfactants), and Alkyl poly glucosides (sold as Plantacare surfactants).

Water is a preferred carrier in liquid cleansing compositions of the invention. In such compositions, water is generally present in 70 to 95% by weight. Preferred liquid cleansing compositions may include other known ingredients such as electrolytes, perfumes, pigments, preservatives, emollients, sunscreens, emulsifiers, gelling agents and thickening agents.

Another aspect of the present invention relates to a method of providing energizing glow to skin comprising the step of applying the composition of the invention on to skin. The method is preferably cosmetic or non-therapeutic.

Yet another aspect of the present invention relates to use of vitamin C or its derivative to boost ATP levels in HDFa cells. Another aspect relates to use of vitamin C or its derivative to provide energizing glow to skin. Yet another aspect of the present invention relates to use of a composition of the present invention for providing energizing glow to skin.

The invention will now be illustrated with the help of the following non-limiting examples.

### EXAMPLES

### Examples A-C, 1: Increase in ATP Levels on Use of Various Compositions

Compositions as shown in Table-1 below were prepared.

TABLE 1

| | Example- | Example- | Example- | Example- |
|---|---|---|---|---|
| Ingredients | 1 | A Placebo | B | C |
| Pemulen TR-1* | 0.08 | 0.08 | 0.08 | 0.08 |
| Glycerine | 5.0 | 5.0 | 5.0 | 5.0 |
| Polysorbate 20** | 1.0 | 1.0 | 1.0 | 1.0 |
| Sodium ascorbyl | 0.1 | — | — | 0.1 |
| phosphate | | | | |
| Niacinamide | 1.0 | 1.0 | 1.0 | 1.0 |
| Olivine Extract | 0.5 | — | 0.5 | — |
| Phenoxyethanol | 0.4 | 0.4 | 0.4 | 0.4 |
| Cremaphore RH 410 | 1.1 | 1.1 | 1.1 | 1.1 |
| Fragrance | 0.48 | 0.48 | 0.48 | 0.48 |
| Water | To 100 | To 100 | To 100 | To 100 |

*Pemulen TR-1 is an acrylates/C10-30 alkyl acrylates crosspolymer
**Polysorbate 20 is also known as Tween 20 and is a non-ionic surfactant

The compositions as shown in Table-1 were subjected to an invitro assay where the increase of ATP in HDFa cells was measured. The assay protocol is given below:

Fibroblast (HDFa cells) culture: Human dermal fibroblast from adult donor (HDFa) were obtained from Lonza and cultured in DMEM media with 10% FCS and antibiotics at 5% CO₂/37° C. When the cells reach 85%, confluence media was removed, cells were washed with 1× PBS and were incubated with 3 ml of trypsin EDTA for 5 min. Trypsin was inactivated by addition of 2 ml trypsin neutralizer. Separated cells were collected and centrifuged down, cells were re-suspended in media and about 2×10\hat{ }5 cells were seeded into fresh cell culture flasks and maintained in a 5% CO₂ incubator at 37° C. Cultures were left undisturbed for 48 h, after which they were fed every 48 h until confluent.

### Estimation of ATP Levels in HDFa Cells:

HDFa cells (10,000 cells/well) were seeded in 96 well plate in DMEM media with 10% FCS and incubated at 5% CO₂/37° C. for 24 h, after 24 h actives were added in DMEM media with 2% FCS and incubated for 1 h/24 h at 5% CO₂/37° C. After incubation cells were lysed by adding 30 μl of RIPA buffer with protease and phosphatase inhibitor. Estimation of ATP levels was done according to kit protocol Molecular probes ATP Determination Kit (A22066) and normalized with protein content estimated by BCA method.

The % ATP levels with respect to the placebo is given in Table-2 below:

TABLE 2

| ATP | Example- | Example- | Example- | Example- |
|---|---|---|---|---|
| levels (%) | 1 | A Placebo | B | C |
| | 133 | 100 | 101 | 107 |

The data in the Table-2 above indicates that Olivine Extract and vitamin C interact synergistically to boost ATP levels thereby providing for a composition that delivers energizing glow to skin.

### Examples a, D,E, 2: Increase in ATP Levels on Use of Individual Actives and their Combination

Actives as shown in Table-3 below were used to measure the increase in ATP at end of 1 hr. The data is also summarized in the same table.

TABLE 3

| | Example A | Example- | Example- | Example- |
|---|---|---|---|---|
| Ingredients | (placebo) | D | D | 2 |
| Olivine Extract | — | 0.001% | — | 0.001% |
| Stay C | — | — | 0.0001% | 0.0001% |
| ATP levels (%) | 100 | 122 | 153 | 220 |

Stay C is a commercially available sodium ascorbyl phosphate from DSM.

The data in the Table-2 above also indicates that Olivine Extract and vitamin C interact synergistically to boost ATP levels as pure actives in the absence of other compositional ingredients.

1. A topical composition comprising:
olivine extract;
vitamin C or its derivative; and
a cosmetically acceptable vehicle selected from (i) water thickened with a polymer or (ii) an emulsion.

2. The composition as claimed in claim 1, wherein the composition comprises from 0.01 to 3 wt % olivine extract.

3. The composition as claimed in claim 1, wherein the composition comprises from 0.01 to 2 wt % vitamin C or its derivative.

4. The composition as claimed in claim 1, wherein the composition is in lotion, cream, gel, serum, or essence form.

5. The composition as claimed in claim 4, wherein the composition is in serum or essence form.

6. The composition as claimed in claim 5, wherein the composition comprises from 55 to 99 wt % water.

7. The composition as claimed in claim 1, wherein the composition comprises from 0.01 to 2 wt % of the polymer selected from one or more of an acrylate/alkyl acrylate cross polymer, carbomer, ammonium acryloyldimethyltaurate/beheneth-25 methacrylate cross-polymer, and ammonium acryloyldimethyltaurate/VP copolymer.

8. The composition as claimed in claim 1 further comprising a polyhydric alcohol selected form one or more of glycerine, 1,3-butylene glycol, propylene glycol, 1,3-propanediol, pentylene glycol, hexylene glycol, and sorbitol.

9. The composition as claimed in claim 1 further comprising a skin brightening compound selected from a vitamin B3 compound, a resorcinol, hydroxystearic acid, pyridoxine hydrochloride, AHA, BHA, PHA, or natural skin brightening extracts like honey or lemon extracts.

10. The composition as claimed claim 1 further comprising moisturizing agents selected from one or both of hyaluronic acid and sodium L-PCA.

11. A method of providing energizing glow to skin comprising the step of applying a composition as claimed in claim 1 on to skin.

12. (canceled)

13. (canceled)

14. The composition as claimed in claim 9, wherein the resorcinol is ethylresorcinol or hexylresorcinol.

15. The composition as claimed in claim 1 further comprising one or more C10 to C22 fatty acid.

16. The composition as claimed in claim 15, wherein the one or more C10 to C22 fatty acid is selected from stearic acid, palmitic acid, hystric acid, and a combination thereof.

17. The composition as claimed in claim 1 further comprising one or more emollients selected from stearyl alcohol, glyceryl monoricinoleate, mink oil, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and combinations thereof.

18. The composition as claimed in claim 1 further comprising a sunscreen.

19. The composition as claimed in claim 1 further comprising a surfactant selected from an anionic surfactant, a non-ionic surfactant, a cationic surfactant, an amphoteric surfactant, and combinations thereof.
